(19) United States
(12) Patent Application Publication
(10) Pub. No.: US 2024/0157072 A1
(43) Pub. Date: May 16, 2024

(54) Capture System

(71) Applicant: Sagetech Medical Equipment Limited, Paignton Devon (GB)

(72) Inventor: Stephen Wileman, Paignien (GB)

(73) Assignee: Sagetech Medical Equipment Limited, Paignton Devon (GB)

(21) Appl. No.: 18/281,521

(22) PCT Filed: Mar. 11, 2022
(86) PCT No.: PCT/EP2022/056415

§ 371 Date: Sep. 11, 2023

(30) Foreign Application Priority Data
Mar. 12, 2021 (GB) 2103489.7
Aug. 24, 2021 (GB) 2112115.7

(51) Int. Cl. A61M 16/00; A61M 16/01; A61M 16/20

(52) U.S. Cl. CPC A61M 16/0093; A61M 16/0012; A61M 16/01; A61M 16/20

(57) ABSTRACT

An anaesthetic gas capture rig (10) for extracting anaesthetic gas from an anaesthesia machine is provided. The rig comprises or can be connected to suction means for drawing gas exhausted from a machine to the rig. The rig is pneumatically decoupled from the machine so that the rig cannot apply vacuum pressure to the machine.

The present invention relates to generally to the capture of halocarbons and more particularly, although not exclusively, to apparatus for use in the capture of volatile anaesthetic agents in medical environments.

A halocarbon is an organic chemical molecule composed of at least one carbon atom bound covalently with one or more halogen elements. Halocarbons have many uses and are used in several industries as solvents, pesticides, refrigerants, fire-resistant oils, ingredients of elastomers, adhesives and sealants, electrically insulating coatings, plastics and anaesthetics. An alternative term for halocarbons is “halogenated fluorocarbons” when halogen elements other than fluorine are included in the molecule.

Volatile anaesthetic agents are typically halogenated fluorocarbons, examples of which include desflurane, isoflurane, sevoflurane and halothane. Volatile anaesthetic agents are liquid at room temperature but evaporate easily to produce a vapour for inhalation by a patient to induce anaesthesia. Anaesthetic agents are used extensively in modern healthcare and represent a significant cost. They are also potent greenhouse gases due to their ability to absorb infrared light and their upper atmospheric persistence. Isoflurane and Halothane also contain Chlorine and Bromine groups that contribute to ozone depletion.

Examples of halocarbons which are used as anaesthetic agents typically include desflurane, isoflurane, sevoflurane, halothane and enflurane. These anaesthetics may be referred to as volatile anaesthetic agents because they are liquid at room temperature but evaporate easily to produce a vapour for inhalation by a patient to induce anaesthesia. These agents are administered to patients using the breathing circuit of an ananaesthetic machine, also known as a Boyle's machine. The primary function of the anaesthetic machine is to mix oxygen with volatile anaesthetic agent, at a clinician-specified concentration, for delivery to the patient via the breathing circuit.

The present invention seeks to provide improvements in or relating to the capture of anaesthetic agents from medical environments.

An aspect of the present invention provides an anaesthetic gas capture rig for extracting anaesthetic gas from an anaesthesia machine, the rig comprises or can be connected to suction means for drawing gas exhausted from a machine to the rig, the rig is pneumatically decoupled from the machine so that the rig cannot apply vacuum pressure to the machine.

An aspect of the present invention provides an anaesthetic gas capture rig for extracting anaesthetic gas from the exhaust of an anaesthesia machine, the rig is connected or connectable to an anaesthesia machine, the rig comprises or is connectable to a suction means for causing gas flow from the machine through the rig, the rig comprises or can be connected to an air break for pneumatically decoupling the rig from the machine so that a generally constant volumetric gas flow is maintained regardless of pneumatic variation caused by the machine or the rig.

The rig may also be connected or connectable to a gas scavenging system, for example an Anaesthetic Gas Scavenging System (AGSS) in a hospital.

The rig may be pneumatically decoupled from the scavenging system.

In some scenarios, therefore, the rig sits between an anaesthetic machine and a scavenging system; it is decoupled on either side so its presence has no pneumatic effect on the overall system.

In some embodiments pneumatic decoupling at either or both sides of the device is achieved by a physical gap and/or by conduits/pipes/hoses with vent holes. For example, in some embodiments the air exhaust line from an anaesthetic machine terminates in a reservoir (e.g. a bucket) that is open to the atmosphere. Then a suction line from the device collects exhaust gases from the reservoir. Because the anaesthetic gases are typically heavier than air they remain at or sink to the bottom of the reservoir, from where they can be collected. This means that suction from the device can never apply vacuum pressure to the anaesthetic machine or to a patient. Similarly, if the device is connected to a gas scavenging system a pneumatic break is also provided, meaning that the gas scavenging system cannot apply vacuum pressure to the device; this, together with the anaesthetic machine side break, provides a double safety feature. In some embodiments, for example the device may include a line for pushing waste gas to the scavenging system (having passed through capture canister/s). The waste gas line from the device may, for example, feed into a scavenging suction line. An open reservoir may, for example, be provided by having holes/gaps in the waste line and/or suction line. A double air break arrangement is therefore provided.

An aspect provides an anaesthetic gas capture system, the system comprises an extraction device, the device comprises or can be connected to suction means for drawing gas including anaesthetic agents thereinto for extraction, the device is pneumatically decoupled from the source of the gas.

The device may, for example, draw gas from an anaesthetic machine.

The device may, for example, draw from a patient. For example the device may draw from a patient mask. A pneumatic break ensures that the device cannot apply suction directly to the patient.

The rig may comprises or has means for receiving one or more capture canisters through which exhaust gas flows, the canisters contain filter material for capturing anaesthetic agent from the gas flow. For example the rig may comprise one or more bays for receiving removable capture canisters. When a canister becomes full it can be removed.

In some embodiments canisters can be removed/installed during operation of the rig.

A valve system may be used to allow canisters to be removed during operation of the rig i.e. it is not necessary to turn the rig off to remove/install a canister.

The rig may comprise means for determining the initial and/or remaining capacity of a canister.

Some embodiments include the use of one or more capture canisters which receive waste gas. For example, waste gas may be diverted from the exhaust of the anaesthetic machine. The waste gas may flow into an ingress pipe that feeds into a capture canister.

The anaesthetic machine from which the canister receives waste gas may deliver several different types of agent. Accordingly, the canister may collect a mixture of volatile anaesthetic agents.

In some embodiments halocarbons are captured onto a filter material contained in a canister from the exhaust of the anaesthetic machine.

Filter materials may, for example, include Silica (SiO2), zeolites, carbon and modified or unmodified silica-based or cellulosic aerogels.

Some embodiments of the present invention relate to a capture rig for housing one or more capture canisters.

When the filter material in a canister is saturated with agent, the feed of waste gas to the ingress pipe may be terminated and the canister removed from an ingress pipe and/or an egress pipe.

Alternatively or additionally a “hotswap” system may be provided. Canister valves may, for example, be provided to enable hotswapping.

A device formed in accordance with the present invention may be a free-standing unit capable of containing up to four cylinders at a time.

The suction means may comprise one or more of a vacuum source, a venturi pump or a fan.

One or more fans may be provided. The fan/s may be used to regulate flow rate, for example using a fan tachometer.

In some embodiments the suction means comprises a plurality of (for example two) fans. This could, for example, be used to build in redundancy to the system.

The present invention also provides a mobile anaesthetic gas scavenging device comprising a rig as described herein.

Materials may be selected so that the apparatus can cope not only with anesthesia gases but also high concentrations of O₂.

A lifting mechanism may be provided.

Some embodiments relate to a capture rig for housing a plurality of capture canisters.

Some embodiments may work with different types of anaesthetic machines e.g. open reservoir and integral weigh cell.

A bypass arrangement may be provided.

The canister/s may be able to withstand temperatures and/or pressures required for subsequent supercritical fluid extraction of agent bound to the filter material.

Alternatively or additionally a pressure-intolerant sleeve containing filter material for capturing one or more types of anaesthetic halocarbon prior to supercritical fluid extraction may be provided. A pressure-tolerant housing into which the sleeve can be inserted may also be provided so as to permit exposure of the sleeve contents to pressures required for supercritical fluid extraction.

Some aspects embodiments relate to the capture of volatile anaesthetic agent and their separation and purification for the purposes of re-supply. This ‘remanufacture’ process is intended to provide financial and environmental cost savings.

Optional features:
an open reservoir (size to be determined by risk of potential breakthrough)
canisters
Potential duplex fan
position of inlet and outlet
what the user interface could be ⋅ how to indicate ‘no canister’ for hotswapping

Some embodiment may discourage taking out all cylinders in IFU (instructions for use).

Emergency swapping may be addressed during training and reflected in instructions printed on the door of the Unit.

In some circumstances there may be a risk presented by switching on without any cylinders—but if it is necessary to continue in an emergency, operations should happen with full knowledge that venting through AGSS.

There may be an indicator for canister ‘present/not present’; it may be preferable not to rely on the AGSS (as in some systems this may not be present).

Open Reservoir

Avoid/prevent potential of breakthrough of volatile at high O2 flow.

rate. 85l/mins max, fan draw at x litres, 20 seconds.

Trolley 4 Cylinders

The product may be 4 cylinders and mounted on a trolley.

An alternative embodiment may be wall mounted and could, for example, have 2 canisters.

In some embodiment a 4-canister model may be used for both wall and trolley mounted as this may increase the safety margin with respect to back pressure. In view of this, it may be possible to have essentially the same unit, either wall or trolley mounted.

User Interface Format—Simple, could be LED or Small Screen

May be traffic lights.

Could be like a fuel tank, or five bars, or needle gauge, for example.

May be connected to weigh cell so measures weight.

Not interactive, only displays capacity.

May provide for fluorocarbon measuring.

Electronics may reset when canister is replaced.

Canister ‘present/not present’ indicator.

Implement Dual Fan/Duplex

If possible, if does cause other problems or risks Inlet/outlet position and AGSS pipe.

This is a usability question—may be best to design from this point of view e.g. both inlets may be clearly labelled together, with different connections.

AGSS pipework is heavy so near the base may be preferable.

Regulatory Requirements

The capture rig may comply with ISO 13485 Class 1a regulations.

Some embodiments allow fitment of 2 (for example) cannisters in parallel.

Capacity for 4 cylinders rather than 2 may be provided.

Inlet/outlet ports may be located at the bottom of the product.

Duplex fans may be provided.

A user interface may be provided.

The apparatus may be provided as a floor standing product.

Regarding the gas flow, the open reservoir may be removed but with scope to fit the part using the same mouldings. In some embodiments the proposal is that this port can be ‘capped’ on early release products, if there is a future requirement for an open reservoir to work in conjunction with anesthesia machines operating in a passive setup (anesthesia machine has no open reservoir) this part could be retrofitted with little impact. This update may be implemented. In >90% of cases the anesthesia machine will be fitted with an open reservoir. Two open reservoirs in series would not allow for extraction of gas.

Different aspects and embodiments of the invention may be used separately or together.

The present invention is also described, by way of example, with reference to the accompanying drawings.

All orientational terms, such as upper, lower, radially and axially, are used in relation to the drawings and should not be interpreted as limiting on the invention or its connection to a closure.

Example embodiments are described in sufficient detail to enable those of ordinary skill in the art to embody and implement the systems and processes herein described. It is important to understand that embodiments can be provided in many alternate forms and should not be construed as limited to the examples set forth herein.

Accordingly, while embodiments can be modified in various ways and take on various alternative forms, specific embodiments thereof are shown in the drawings and described in detail below as examples. There is no intent to limit to the particular forms disclosed and as well as individual embodiments the invention is intended to cover combinations of those embodiments as well. On the contrary, all modifications, equivalents, and alternatives falling within the scope of the appended claims should be included. Elements of the example embodiments are consistently denoted by the same reference numerals throughout the drawings and detailed description where appropriate.

The terminology used herein to describe embodiments is not intended to limit the scope. The articles “a,” “an,” and “the” are singular in that they have a single referent; however, the use of the singular form in the present document should not preclude the presence of more than one referent. In other words, elements referred to in the singular can number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof.

Unless otherwise defined, all terms (including technical and scientific terms) used herein are to be interpreted as is customary in the art. It will be further understood that terms in common usage should also be interpreted as is customary in the relevant art and not in an idealized or overly formal sense unless expressly so defined herein.

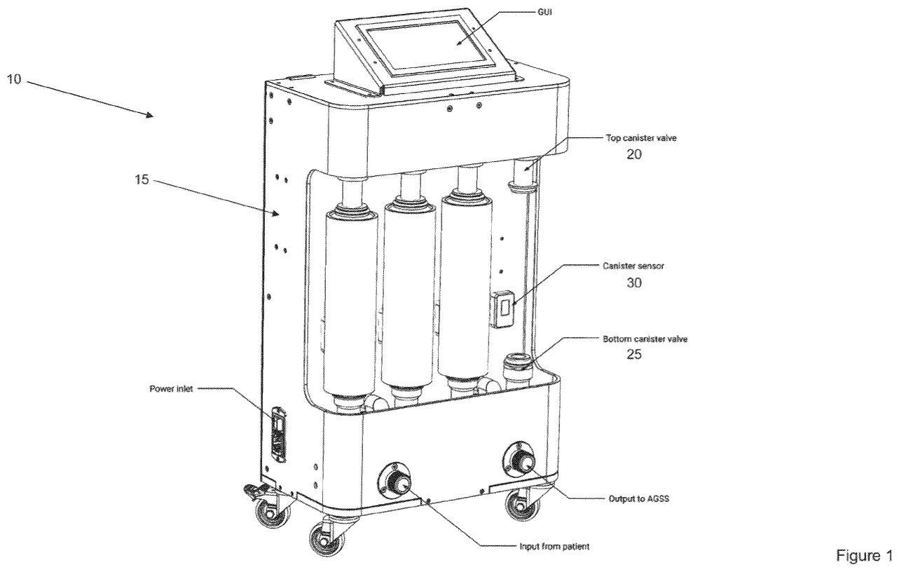

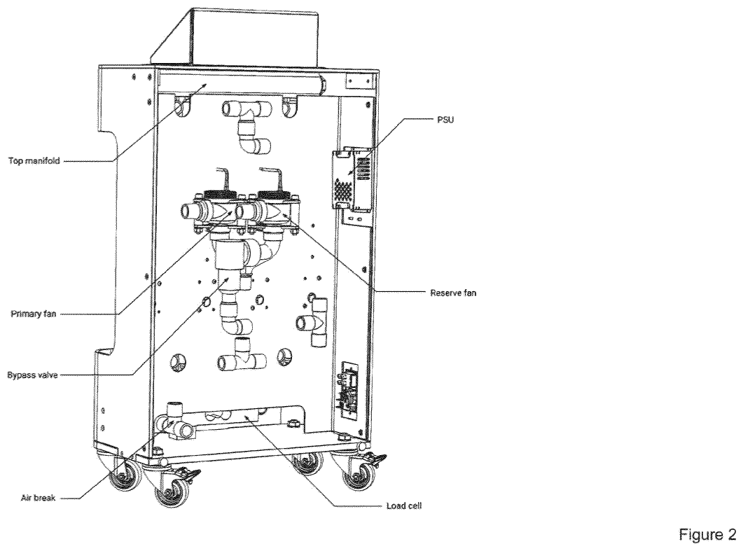

FIGS. 1 and 2 show a capture rig 10 formed in accordance with the present invention and comprising a main body 15. The body 15 has four (in this embodiment) pairs of top and bottom canister valves 20, 25. Canister sensors 30 are provided to detect the presence/absence of a canister in each canister bay. A graphical user interface (GUI) is provided on the body.

The anaesthetic gas capture rig 10 extracts anaesthetic gas from an anaesthesia machine. The rig comprises or can be connected to suction means for drawing gas exhausted from a machine to the rig. The rig is pneumatically decoupled from the machine so that the rig cannot apply vacuum pressure to the machine.

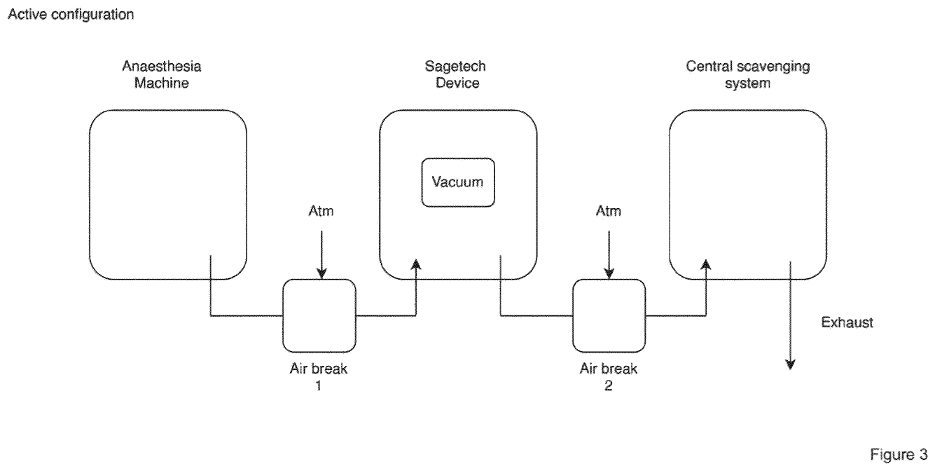

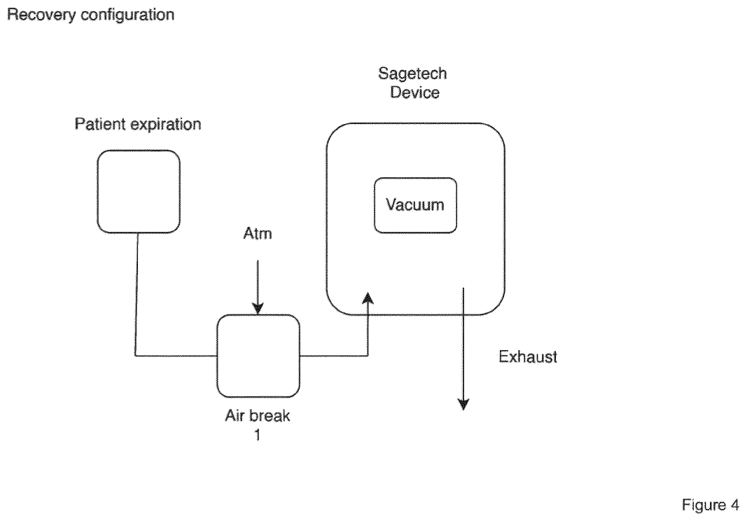

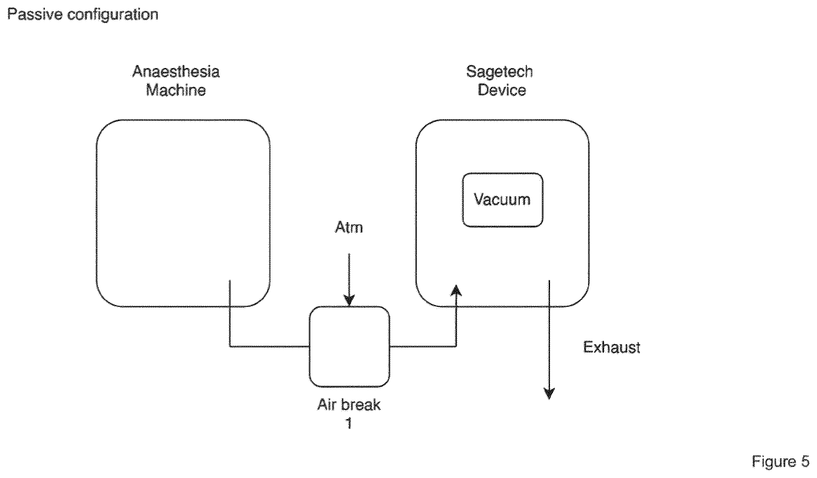

FIGS. 3, 4 and 5 show schematics for Active, Passive and Recovery configurations of aneasthesia systems including capture rigs (“Sagetech Device”) formed in accordance with the present invention.

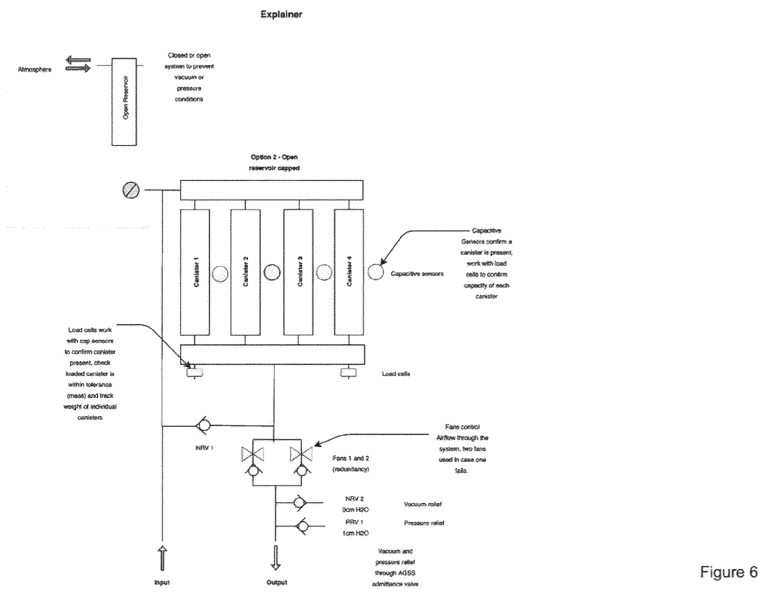

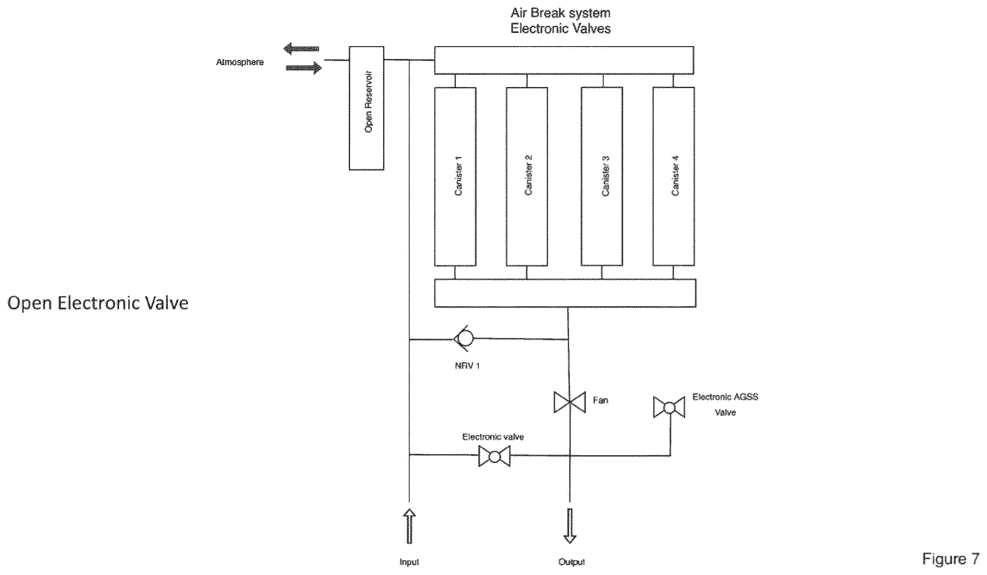

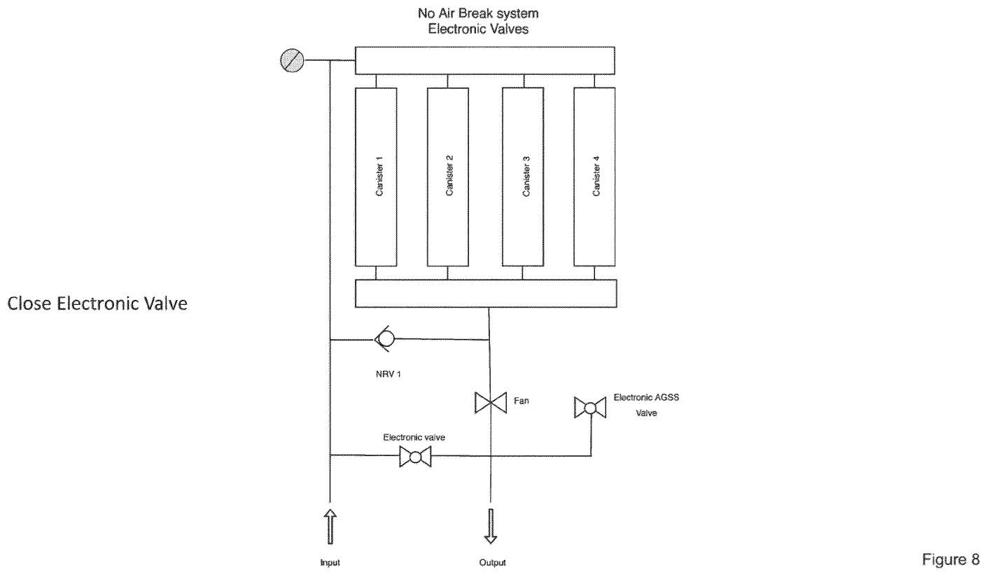

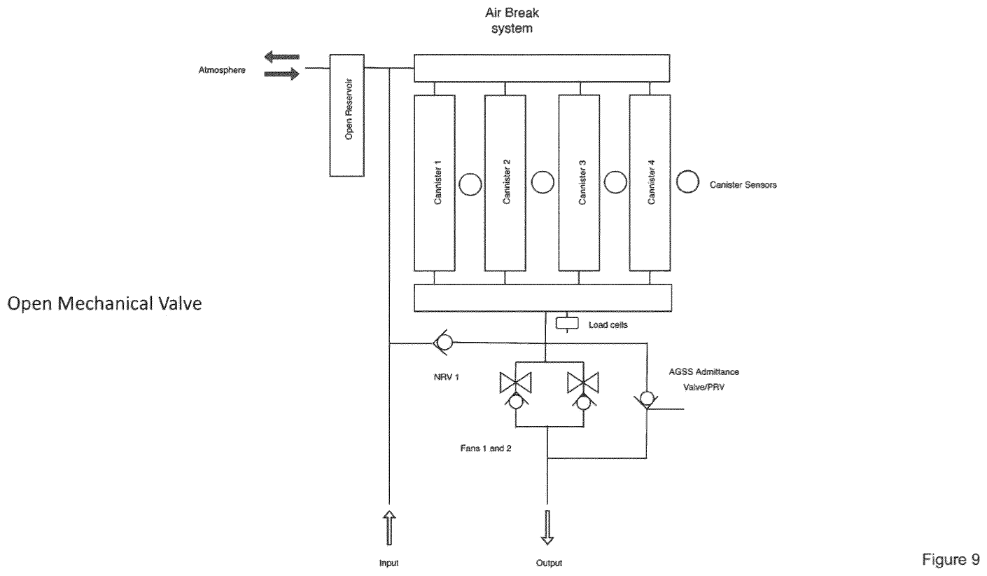

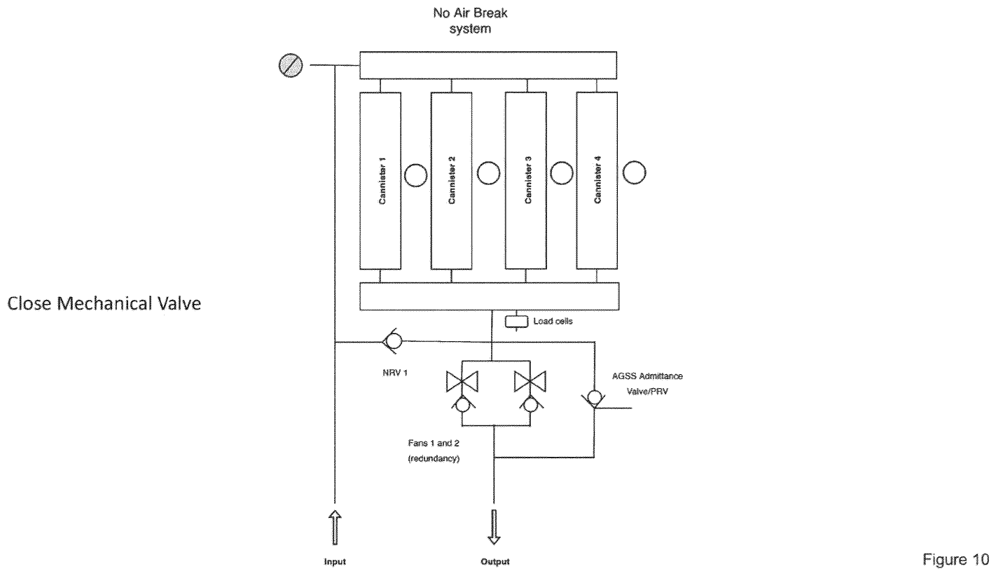

FIGS. 6 to 10: explanation and detailed schematics for open and closed systems in both mechanical and electronic configurations.

In the schematics the rig has been shown with a vacuum source. This could be an external or internal vacuum source, venturi pump, or fans. The vacuum pressure may be between 10-20 kPa vacuum pressure with a flow rate between 50-80 lpm, for example. These values could increase or decrease if there are changes to legislation.

Either side of the device is an air break that effectively de-couples each of the systems so no over-pressure or vacuum condition can affect the patient.

The device is fitted with valves that allow canisters to be swapped while in operation. In a condition where all canisters have been removed, the system would move to a bypass condition so there is no interruption to gas flow through the systems.

Load cells and canister sensors work together to determine the presence and mass of individual canisters so we can track their capacity and indicate when they need to be replaced.

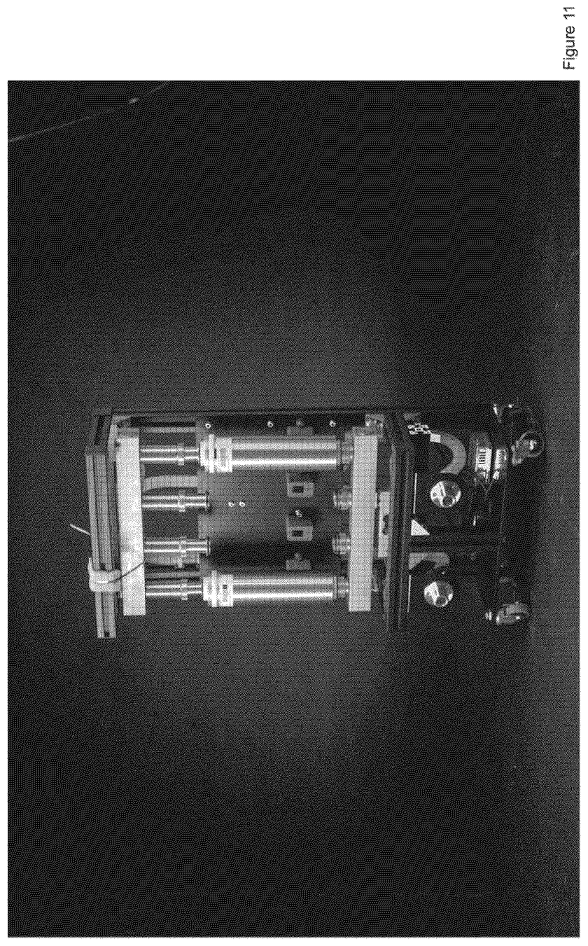

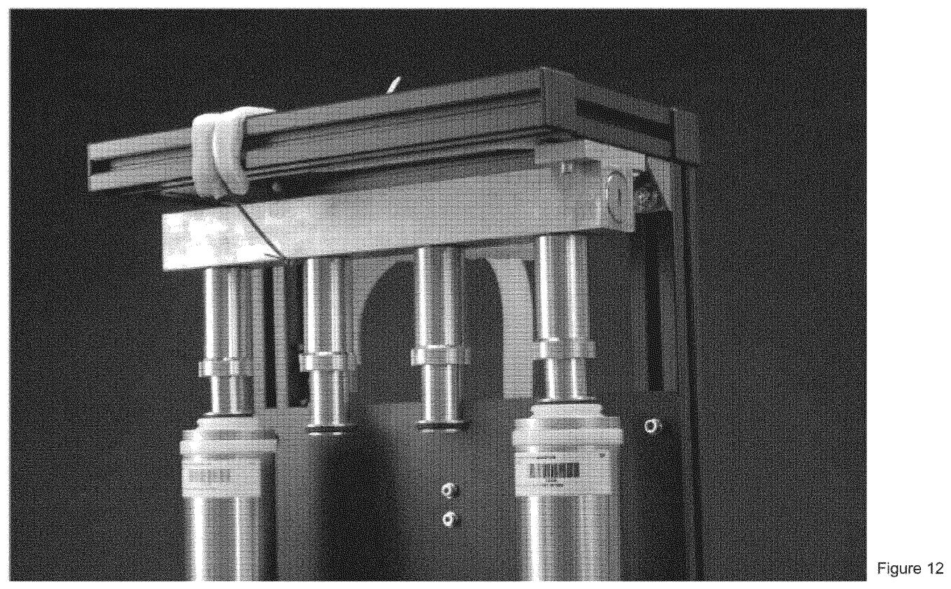

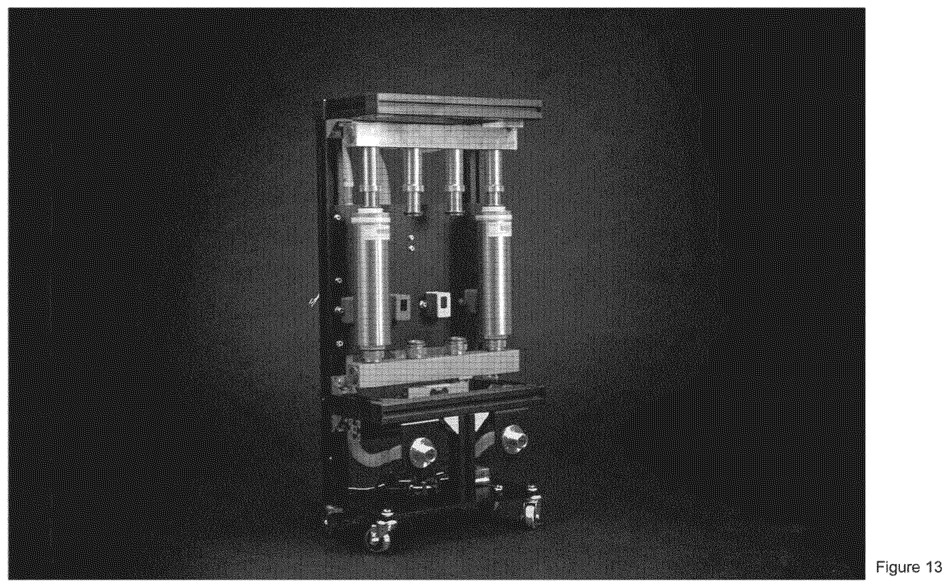

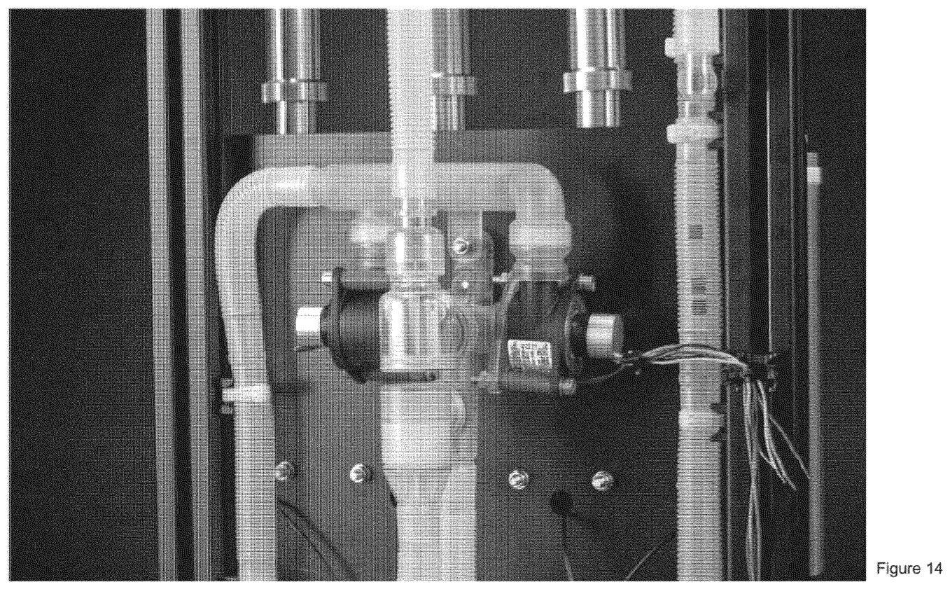

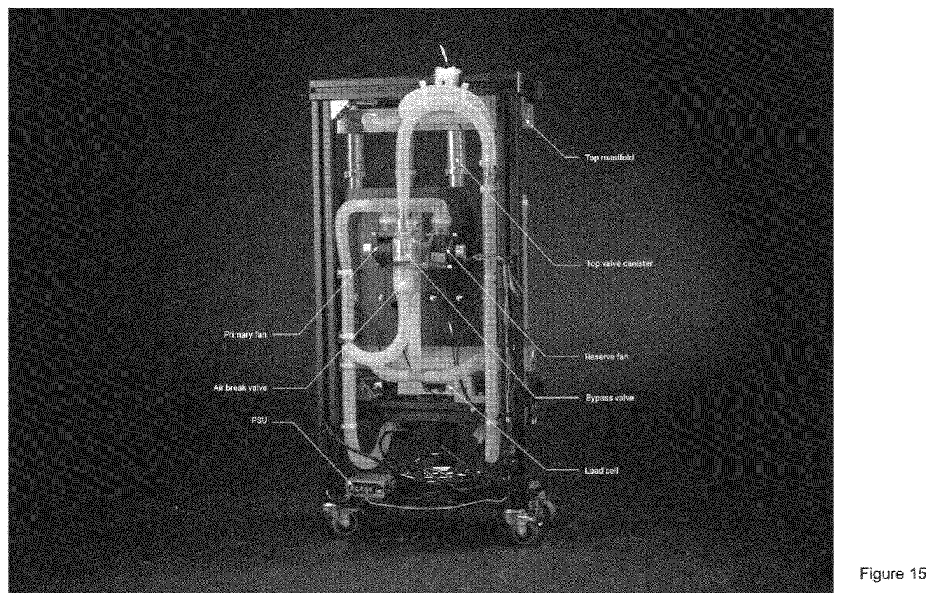

FIGS. 11 to 15 illustrate a capture rig 110 formed in accordance with the present invention.

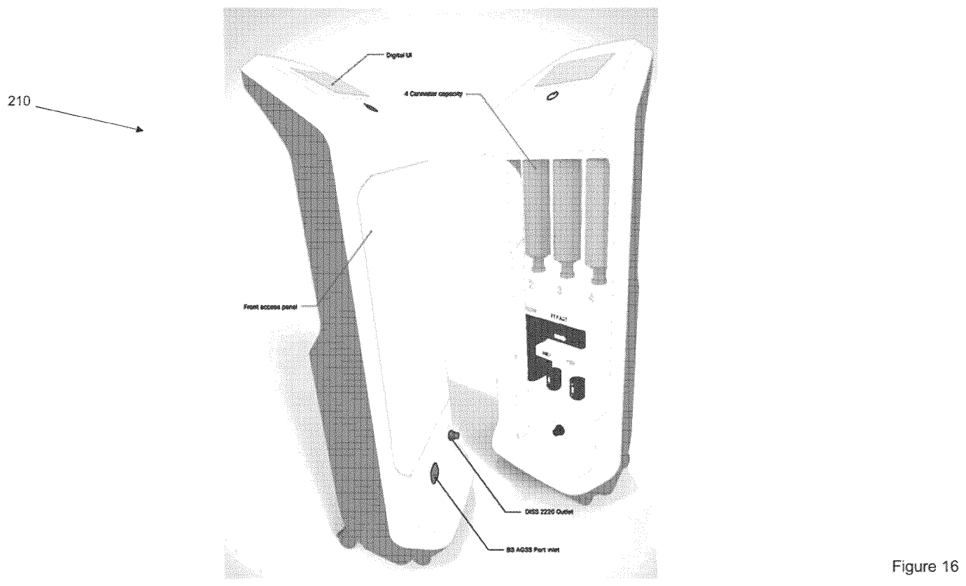

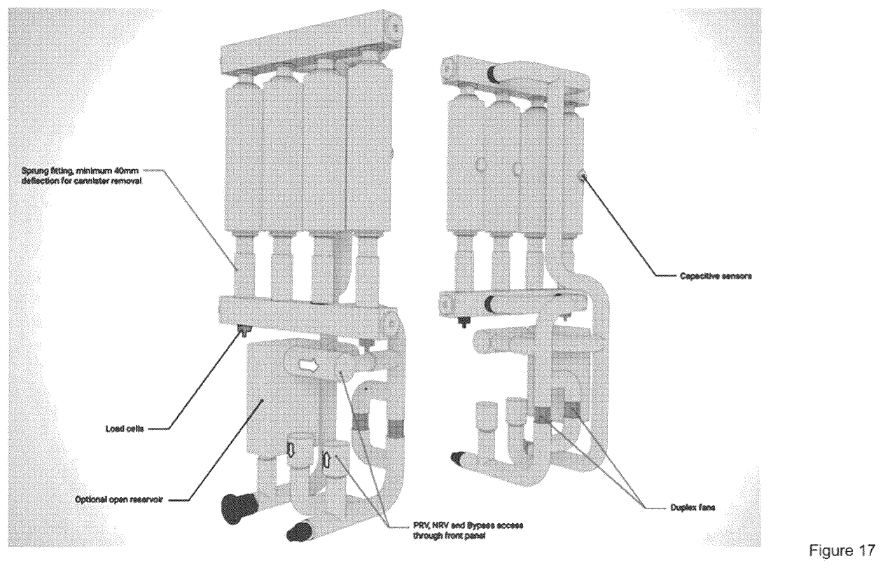

FIGS. 16 and 17 show a capture rig 210 formed according to a further embodiment.

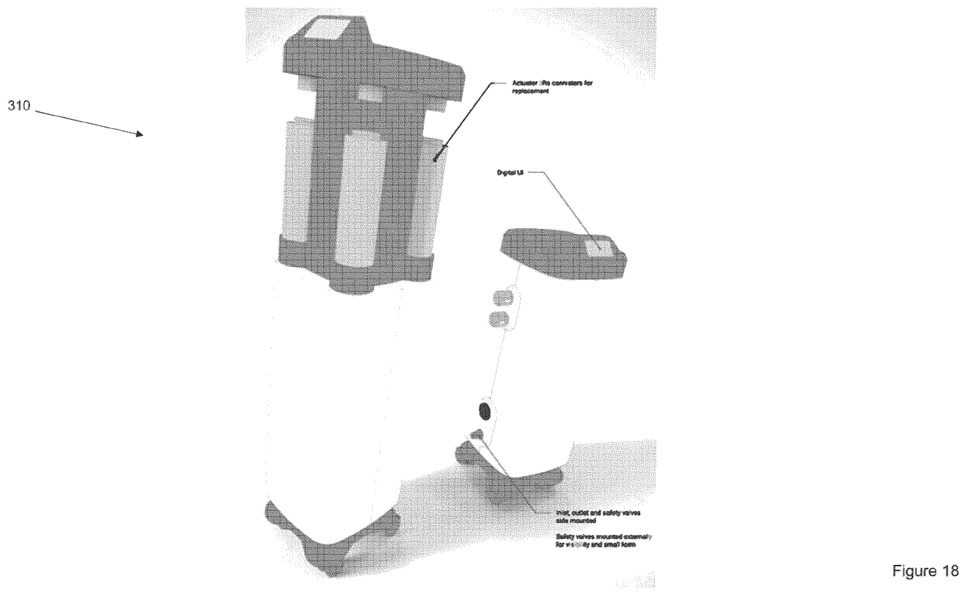

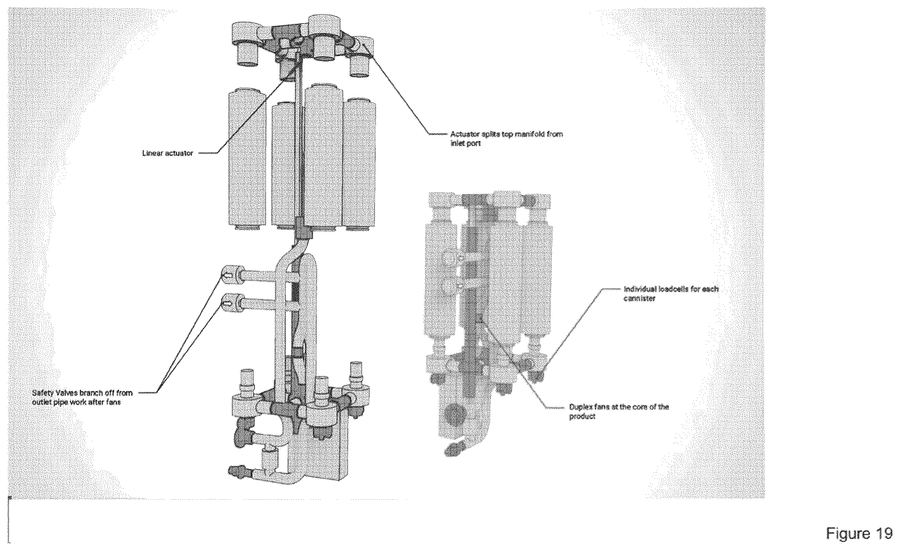

FIGS. 18 and 19 show a capture rig 310 formed according to a further embodiment.

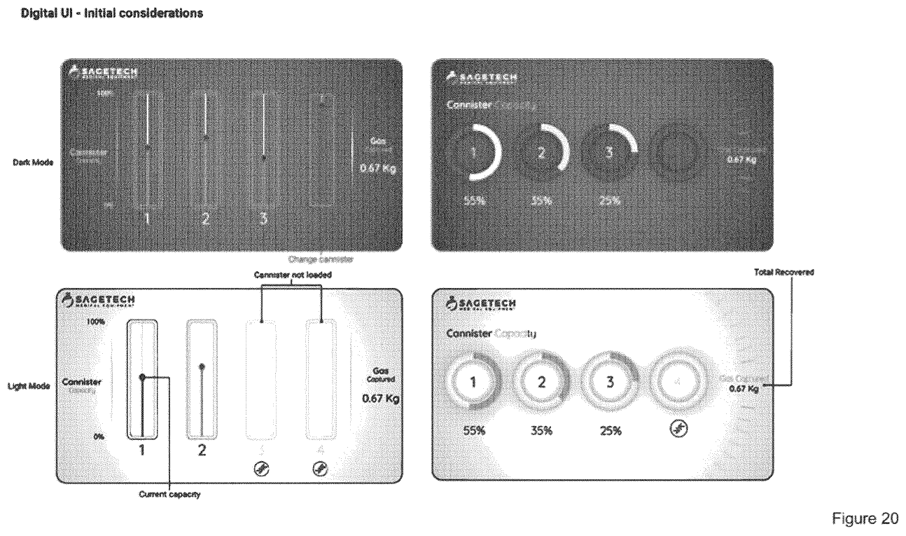

FIG. 20 shows examples of different user interface options.

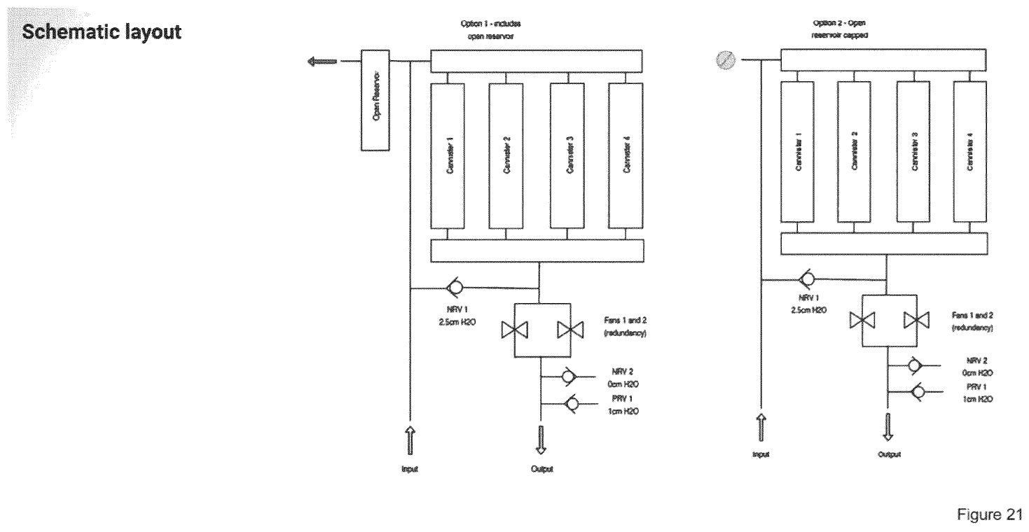

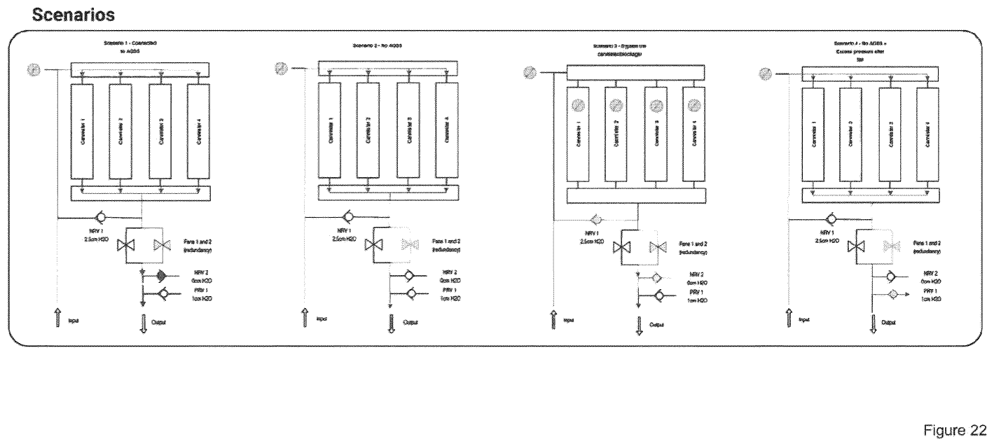

FIGS. 21 to 31 show different schematics to illustrate different scenarios, modes of operation for embodiments of the present invention.

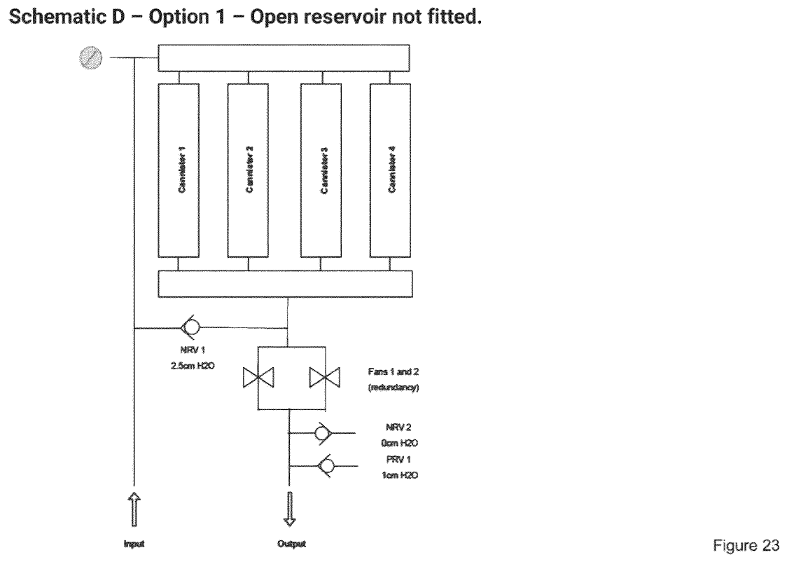

Schematic D—Option 1—Open reservoir not fitted (FIG. 23)

In this case the open reservoir fitted to the anesthesia machine will assume the role of our reservoir, so it is not required. NRV1 is the bypass positioned directly between the inlet and exhaust ports to the product, the bypass must be positioned before the fans on the outlet. Fans 1 and 2 shown to deal with the requirement for redundancy. NRV2 is effectively and air admittance valve which serves the AGSS, gas will be driven through the system by the fan into the AGSS air stream if present. If AGSS is not present, gas will be driven to the exhaust solely by the fans. There is no requirement for the user to make any changes to the system in either scenario. PRV1 is present to vent in case of positive pressure, this could be caused by failure to fit an exhaust pipe or a buckled exhaust pipe. CM H2O values are indicative.

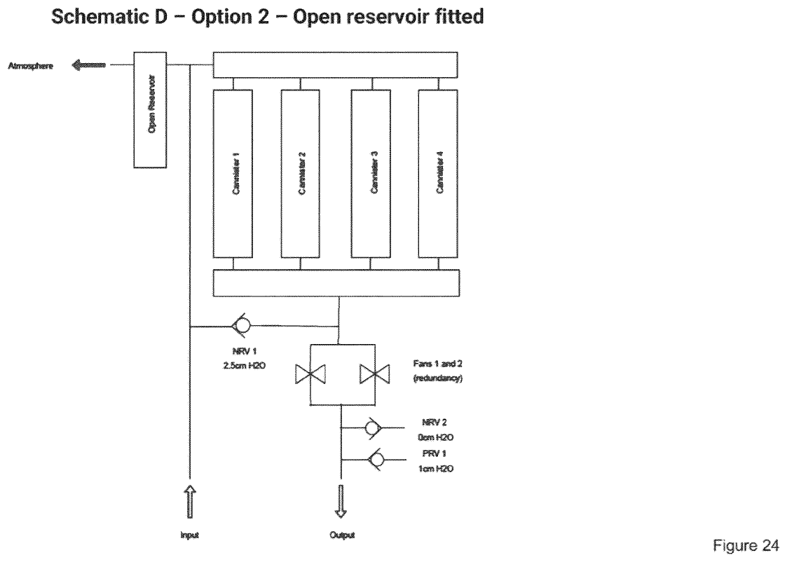

Schematic D—Option 2—Open reservoir fitted (FIG. 24)

Option 2 illustrates the fitment of the open reservoir for use with ‘passive’ setups. As mentioned previously, the open reservoir in this case will deal with O2 flush and fan draw to ensure no impact on the anesthesia machine. The rest of the system remains the same and so supports retrofitting of an open reservoir ‘cartridge’.

The schematic layouts on the following pages detail how the system could cope with various scenarios. These could be a result of cannister removal, blockage or incorrect use. The system should always fail safe. These scenarios have been illustrated with no open reservoir fitted to the device; green lines represent anesthesia gas flow.

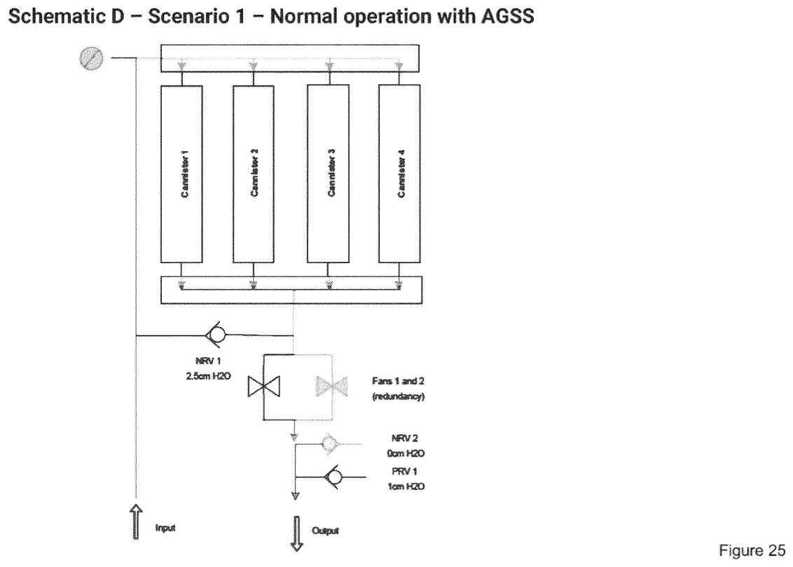

Schematic D—Scenario 1—Normal operation with AGSS (FIG. 25)

AGSS draws through NRV 2, Fan draws gases through the cannisters and into the AGSS air stream.

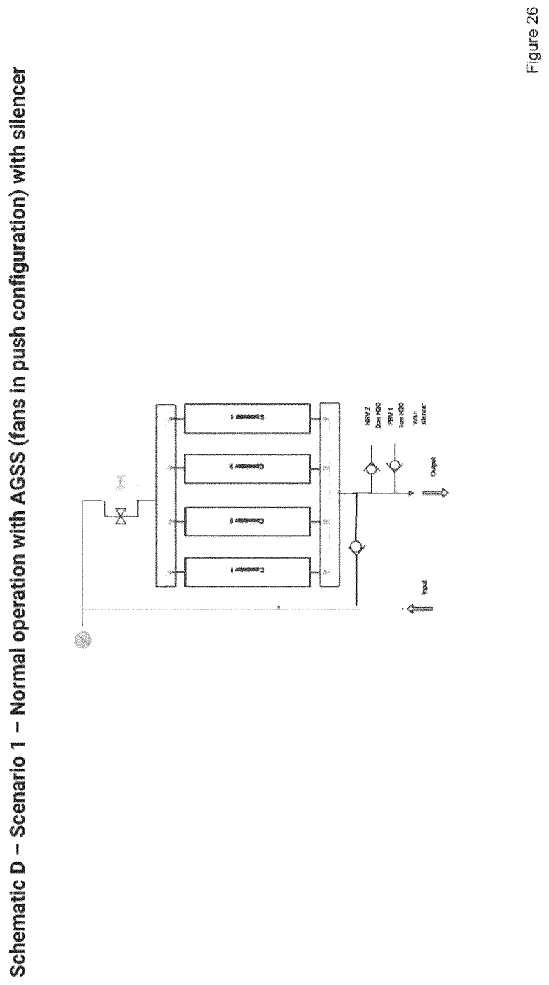

Schematic D—Scenario 1—Normal operation with AGSS (fans in push configuration) with silencer (FIG. 26). The fan is positioned at the top of the canister, “pushing” the gas rather than “pulling”). A silencer is included.

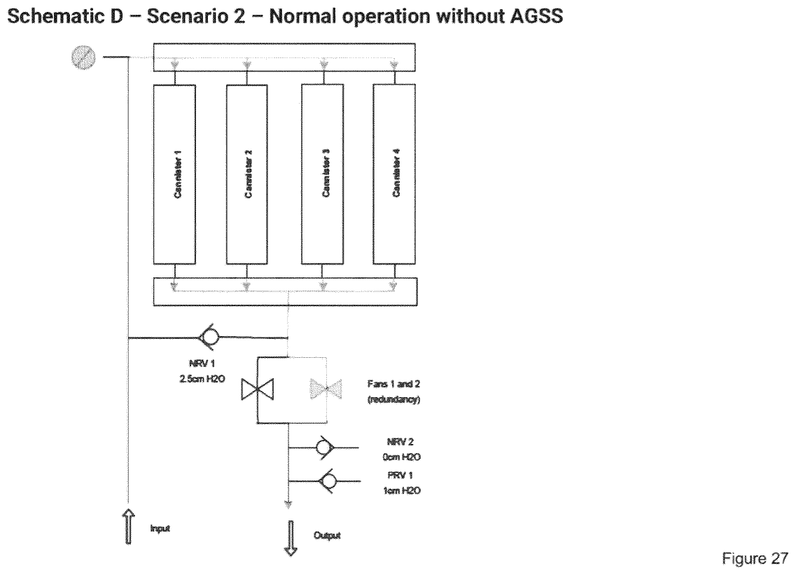

Schematic D—Scenario 2—Normal operation without AGSS (FIG. 27).

Fans draw gases through the cannisters and out to exhaust.

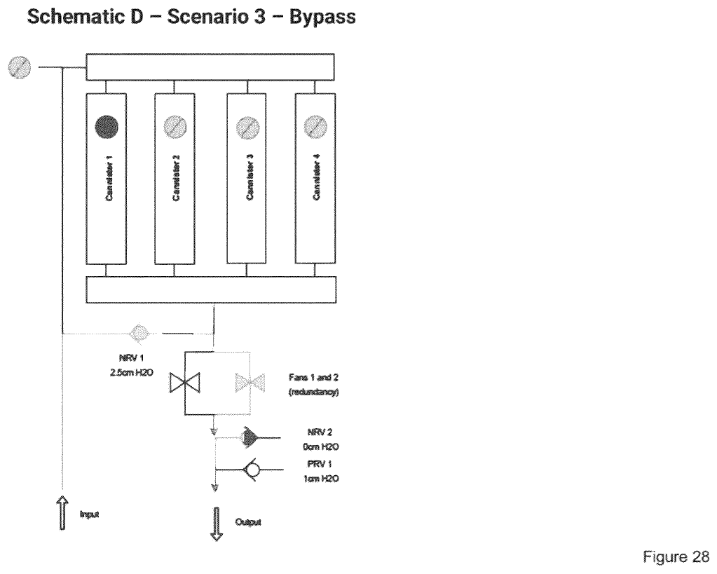

Schematic D—Scenario 3—Bypass (FIG. 28).

Gases bypass cannisters and run directly to exhaust from the input, this scenario could occur because of cannister removal, blockage or failure to fit exhaust pipe.

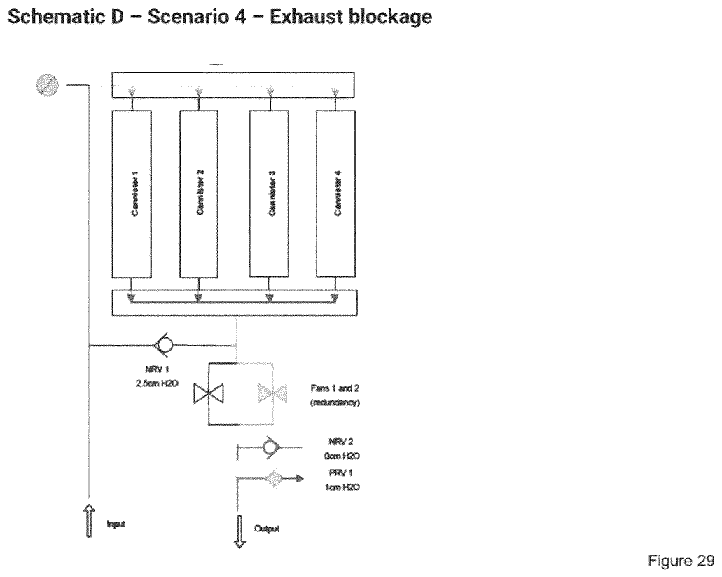

Schematic D—Scenario 4—Exhaust blockage (FIG. 29).

Gases exit through PRV1 (to local environment) in the event of blockage on exhaust. Causes—failure to fit exhaust pipe, bucked pipe.

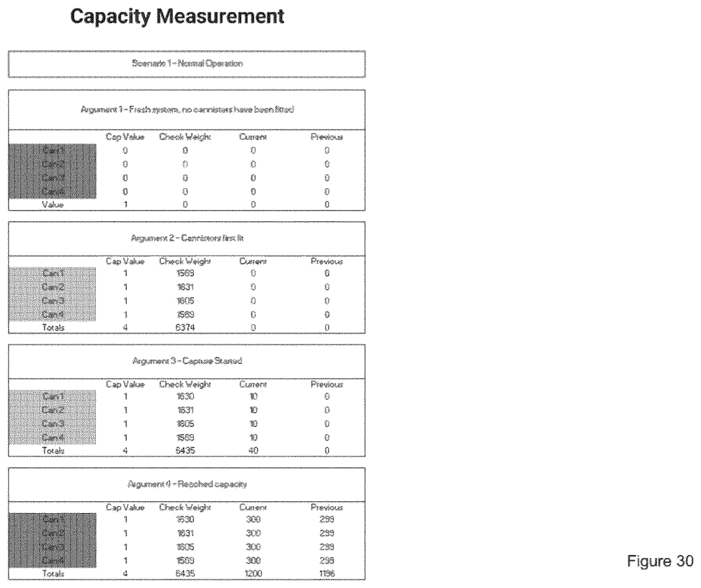

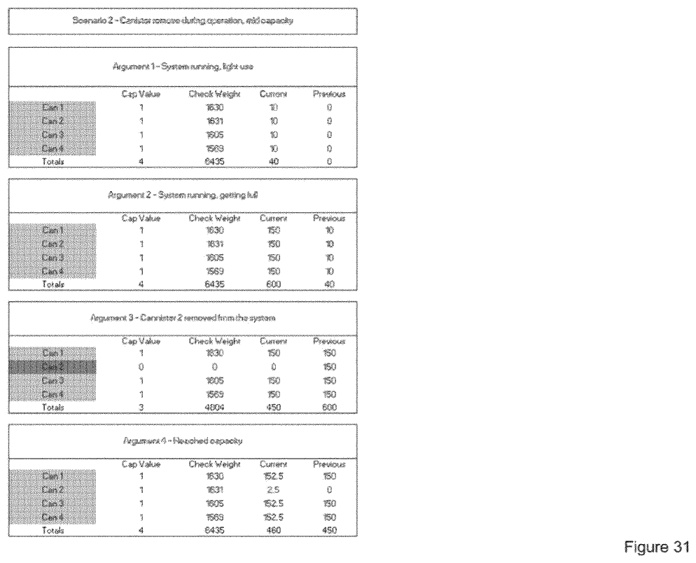

Capacity Measurement—FIGS. 30 and 31.

The proposed concepts make use of load cells and capacitive sensors to check the capacity, Concept B is slightly different in that we have the ability to ‘split’ the manifold and effectively float each cannister to take individual readings. Concept A operates slightly differently but the same logic could be applied to Concept B.

The idea is that the capacitive sensor can be used to verify the removal or replacement of a cannister, it would require a reading from both the capacitive sensor and load cell to confirm canisters have been loaded correctly. Looking deeper into the logic of this system, it is also possible that we could track the capacity of individual cannisters. This is explained in more detail below.

In this scenario, the system starts with no cannisters fitted. The user comes along and fits 4 cannisters. Each of these cannisters will have a tolerance on their mass, for the purpose of this illustration we have assumed a fairly large tolerance of 2.00%, we will use this tolerance on the full capacity lower limit and empty capacity upper and lower limit.

The first check the system must perform is to confirm the cannister sits within the empty capacity tolerance. If it does, it can set the current capacity counter to 0 and confirm that the cannister has been correctly loaded on the UI. If not, we have the option to reject the cannister or with a little more consideration we could determine the remaining capacity by deducting the empty capacity upper limit value from the measured mass.

The next step is to start measuring, the load cells are now tracking the change in mass and counting up on the current value.

Once the capacity reaches the set limit, the UI can indicate the cannisters require changing. This is all fairly straight forward on a system running with all 4 cannister filling at the same rate.

With the 4 capacitive sensors and load cells working together we also have the ability to track the capacity of individual cells. This could be particularly useful in a scenario where the user has decided to change only 1 cannister (that said, we know this isn't recommended behaviour as this won't encourage the gas to bias empty cannisters but it does illustrate the versatility of the system). See below for more detail.

In this scenario, the user has started running the system with a set of 4 cannister that are almost empty. They have continued to use the system as shown in argument 2 and a previous reading has been recorded.

In argument 2, the capacitive sensor reads 0 to indicate that cannister 2 has been removed. At this point the system knows that the value of load cell has reduced but it also knows that a cannister has been removed. The previous reading of the cannister that's been removed could then be deducted from the previous total so it has no bearing on the running total of the other cannisters.

In argument 4 cannister 2 is replaced, the initial checks will be carried out and the current reading forced to 0. The system will then continue as normal dividing the total load cell reading across all cannisters relative to their previous reading if they're not been removed from the system.

Concept A builds on other embodiments, with the addition of 2 more cylinders, the digital UI and reconfiguration of the inlet and outlet ports.

The digital UI is designed as monitor only, one change the user may wish to make is to switch between light and dark themes. This seems to be typical of monitors placed in theatre particularly for key hole surgery where excess light might be undesirable. An alternative might be to lead with a single setting rather than allowing the user to switch but this can be determined as the product develops.

Access to cannisters is achieved by opening the front panel. The safety valves and cavity for an open reservoir will also be revealed at this point so that users can perform checks ahead of starting a procedure.

Each cannister could be numbered for easy identification of specific cannister if they need to be changed individually. The valve at the base of the cannister should be designed to support the load of the cannister at full capacity plus compressive force of ‘x’ for good seating on the seal. The longer the free length of the spring the lighter the spring we can use to achieve 40 mm travel for cannister removal (Travel distance will have a direct impact on the total height of the device).

The inlet and outlet ports have been moved to the base of the product to cope with the weight of the AGSS pipework. These could use standard BS and DISS fittings, these should be quite familiar to users of the anesthesia machines.

Concept B Is an alternative concept with a very different take on the construction of the product and user experience. The idea is that the cannisters could be accessed by a carrier lifting out of the base of the product, this lifts the cannisters to a comfortable working height. The manifold would split as the linear actuator raises the cannisters (could also be achieved with gas springs or other mechanisms but requires more consideration).

This concept allows for a much smaller former factor than concept A, this is down to routing of internal pipework and presenting the safety valves on the outside of the product rather than concealing them within. Functionality remains the same between both concepts as they rely on the same schematic but the physical arrangement is what differs.

As in concept A, the inlet and outlet ports are positioned low on the product but both the gas ports and safety valves are located on the side rather than front face of the product. This might help with pipe routing within a theatre.

Where the intention on concept A is to display instructions on the inside panel of the door, the same approach could be applied to the flat faces of the carrier as the cannisters are lifted in concept B.

Although illustrative embodiments of the invention have been disclosed in detail herein, with reference to the accompanying drawings, it is understood that the invention is not limited to the precise embodiments shown and that various changes and modifications can be effected therein by one skilled in the art without departing from the scope of the invention.

1. An anaesthetic gas capture rig for extracting anaesthetic gas from an anaesthesia machine, the rig comprises or can be connected to suction means for drawing gas exhausted from a machine to the rig, the rig is pneumatically decoupled from the machine so that the rig cannot apply vacuum pressure to the machine.

2. A rig as claimed in claim 1, in which the rig is also connected or connectable to a gas scavenging system.

3. A rig as claimed in claim 1, in which the rig is also connected or connectable to an Anaesthetic Gas Scavenging System (AGSS).

4. A rig as claimed in claim 2, in which the rig is pneumatically decoupled from the scavenging system.

5. A rig as claimed in claim 1, in which the rig comprises or has means for receiving one or more capture canisters through which exhaust gas flows, the canisters contain filter material for capturing anaesthetic agent from the gas flow.

6. A rig as claimed in claim 5, in which the or each canister can be removed/replaced during operation of the rig.

7. A rig as claimed in claim 5, comprising means for determining the remaining capacity of a canister.

8. A rig as claimed in claim 5, comprising means for detecting the presence of a canister.

9. A rig as claimed in claim 1, in which the suction means is one or more of a vacuum source, a venturi pump or a fan.

10. A rig as claimed in claim 1, in which the suction means comprises a plurality of fans.

11. A rig as claimed in claim 1, comprising an air break formed within or between flow conduits.

12. A rig as claimed in claim 1 and configured as mobile, floor-standing anaesthetic gas scavenging device.

13. An anaesthetic gas capture system for extracting anaesthetic gas from the exhaust of an anaesthesia machine, the system comprises an extraction device, the device comprises or can be connected to suction means for drawing gas exhausted from a machine to the device, the system comprises an open reservoir from which the suction means draws gas, the machine exhausts into the open reservoir, whereby the rig is pneumatically decoupled from the machine so that the device cannot apply vacuum pressure to the machine.

14. A system as claimed in claim 13, in which the device is connected to a gas scavenging system.

15. A system as claimed in claim 14, in which the connection from the device to the scavenging system includes a pneumatic break.

16-19. (canceled)

20. An anaesthetic gas capture device rig as claimed in claim 1, the rig comprises or is connectable to a suction means for causing gas flow from the machine through the rig, the rig comprises or can be connected to an air break for pneumatically decoupling the rig from the machine so that a generally constant volumetric gas flow is maintained regardless of pneumatic variation caused by the machine or the rig.

21. A rig as claimed in claim 1, the rig comprises or can be connected to an air break for pneumatically decoupling the rig from the machine so that a generally constant volumetric gas flow is maintained regardless of pneumatic variation caused by the machine or the rig.

22. A rig as claimed in claim 5, comprising a valve system to allow a canister to be removed or installed during operation.

23. A rig as claimed in claim 5, in which when filter material in a canister is saturated the feed of exhausted gas

24. A rig as claimed in claim 5, comprising a top canister valve and a bottom canister valve.
